(19) United States
(12) Patent Application Publication
(10) Pub. No.: US 2010/0068171 A1
(43) Pub. Date: Mar. 18, 2010

(54) INJECTABLE BONE/POLYMER COMPOSITE BONE VOID FILLERS

(75) Inventors: Scott A. Guelcher, Franklin, TN (US); Andrea E. Hafeman, Nashville, TN (US); Michelle B. Brouner, Indianpolis, IN (US)

Correspondence Address: STITES & HARBISON PLLC, 401 COMMERCE STREET, SUITE 800, NASHVILLE, TN, 37219, US

(73) Assignee: Vanderbilt University

(21) Appl. No.: 12/473,246

(22) Filed: May 27, 2009

Related U.S. Application Data
(60) Provisional application No. 61/056,438, filed on May 27, 2008.

(51) Int. Cl. A61K 31/765; C08J 9/00; A61P 19/00; C08G 18/00

(52) U.S. Cl. 424/78.37; 521/170; 521/94; 521/89; 521/82

(57) ABSTRACT

A biodegradable polyurethane scaffold that includes a HDI trimer polyisocyanate and at least one polyol; wherein the density of said scaffold is from about 50 to about 250 kg m−3 and the porosity of the scaffold is greater than about 70 (vol %) and at least 50% of the pores are interconnected with another pore. The scaffolds of the present invention are injectable as polyurethane foams, and are useful in the field of tissue engineering.

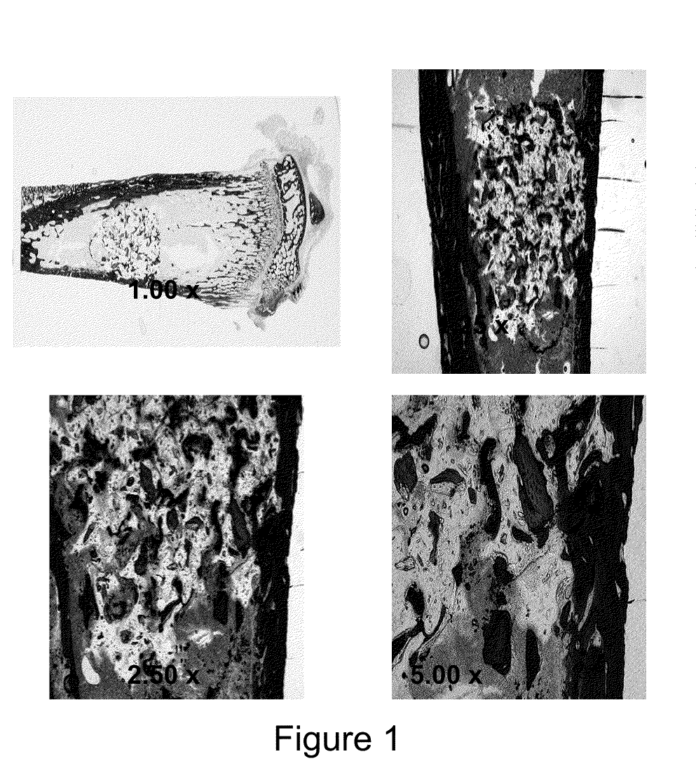

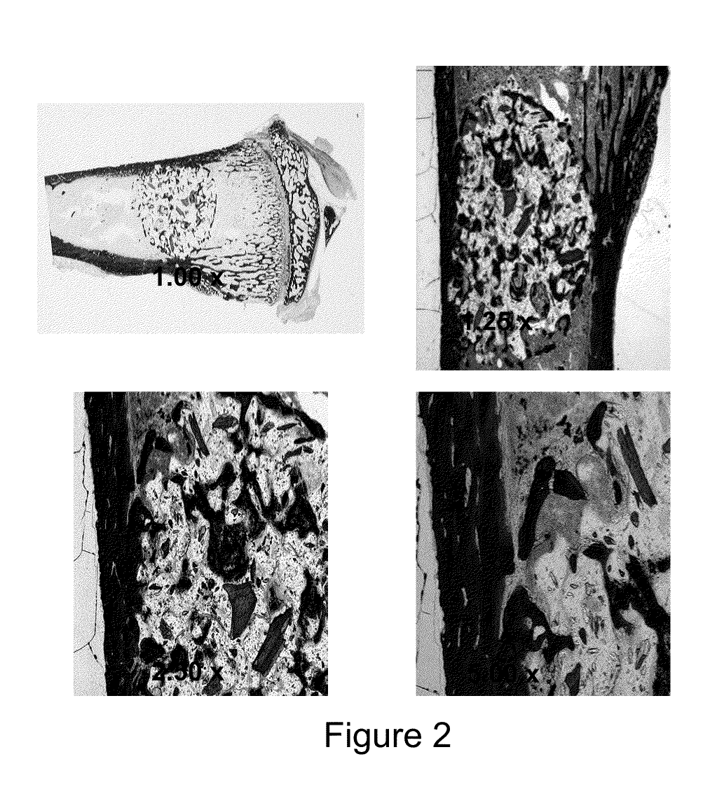

### PRIORITY INFORMATION

This application claims benefit to U.S. Patent Application No. 61/056,438, filed May 27, 2008, the contents of which are incorporated herein by reference in their entirety.

### GOVERNMENT SUPPORT

This invention was made with government support under US Army Institute of Surgical Research Grant No. W81XWH-06-0654, and W81XWH-07-1-0211. The government has certain rights in this invention.

### FIELD OF THE INVENTION

The present invention generally relates to methods and compositions for treatment of bone fractures. The materials are injectable, biodegradable, and contain integrated bone particles to enhance the osteoconductive properties of the foams. Scaffold degradation and release of bioactive components can be controlled independently. Conventional materials, such as tricalcium phosphates, poly(methyl methacrylate), and poly(D,L-lactide-co-glycolide) cannot meet all of these performance requirements.

### BACKGROUND OF THE INVENTION

Due to the high frequency of bone fractures, resulting in over 900,000 hospitalizations and 200,000 bone grafts each year in the United States, there is a compelling clinical need for improved fracture healing therapies. Fractures can result from trauma or pathologic conditions, such as osteoporotic compression fractures and osteolytic bone tumors. Autologous bone grafts are an ideal treatment due to their osteogenic, osteoinductive, and osteoconductive properties, but they are available in limited amounts and frequently result in donor site morbidity. Both synthetic and biological biomaterials have been investigated as substitutes for autogenous bone grafts, and a number of desirable properties have been identified for biomaterials designed for orthopedic applications. Their use can also be extended to soft tissue repair. The biomaterial and its degradation products must be biocompatible and non-cytotoxic, generating a minimal immune response. High porosity and inter-connected pores facilitate the permeation of nutrients and cells into the scaffold, as well as ingrowth of new tissue. Scaffolds should also undergo controlled degradation, preferably at a rate comparable to new tissue formation, to non-cytotoxic decomposition products. Materials that exhibit working times of 3-10 minutes and low temperature exotherms are particularly suitable for clinical use as injectable therapies that can be administered percutaneously using minimally invasive surgical techniques. Additionally, scaffolds should possess sufficient biomechanical strength to withstand physiologically relevant forces. Release of growth factors with fibrogenic, angiogenic, and osteogenic properties, such as platelet-derived growth factor (PDGF), vascular endothelial growth factor (VEGF) and bone morphogenetic protein-2 (BMP-2), may further enhance integration of the device and improved healing.

Due to their ability to meet many of the above-mentioned performance characteristics, both synthetic and biopolymers have been investigated as scaffolds for tissue engineering. The poly(α-esters), including polylactic acid (PLA), polyglycolic acid (PGA), and their copolymers (PLGA), are thermoplastic polymers incorporated in a variety of FDA-approved biomedical devices, including surgical sutures, orthopedic fixation, and drug and growth factor delivery. Scaffolds prepared from other thermoplastic biomaterials, such as tyrosine-derived polycarbonates and polyphosphazenes, have been shown to exhibit tunable degradation to non-cytotoxic decomposition products, high tensile strength, and bone tissue ingrowth in vivo. However, thermoplastic biomaterials cannot be injected, and must be melt- or solvent-processed ex vivo to yield solid scaffolds prior to implantation. Injectable hydrogels, such as poly(ethylene glycol) (PEG), collagen, fibrin, chitosan, alginate, and hyaluronan, have been shown to support bone ingrowth in vivo, particularly when combined with angio-osteogenic growth factors. However, hydrogels lack the robust mechanical properties of thermoplastic polymers.

Two-component reactive polymers are promising scaffolds because they can be formed in situ without the use of solvents. Poly(propylene fumarate) (PPF) can be injected as a liquid and thermally or photo cross-linked in situ with various cross-linking agents, which affect the final mechanical and degradation properties. Recently developed porous composite scaffolds have been formed in situ by gas foaming, with up to 61% porosity, 50-500 μm pores, and a compressive modulus of 20-40 MPa. PPF biomaterials have been shown to support osteoblast attachment and proliferation in vitro, and ingrowth of new bone tissue in vivo. Growth factors have been incorporated via PLGA microspheres into poly(propylene fumarate) materials for controlled release.

Two-component biodegradable polyurethane (PUR) networks have also been investigated as scaffolds for tissue engineering. Porous PUR scaffolds prepared from lysine-derived and aliphatic polyisocyanates by reactive liquid molding have been reported to degrade to non-toxic decomposition products, while supporting the migration of cells and ingrowth of new tissue in vitro and in vivo. However, many polyisocyanates are toxic by inhalation, and therefore polyisocyanates with a high vapor pressure at room temperature, such as toluene diisocyanate (TDI, 0.018 mm Hg) and hexamethylene diisocyanate (HDI, 0.05 mm Hg), may not be suitable for injection in a clinical environment. To overcome this limitation, the present inventors and others have formulated injectable PUR biomaterials using lysine diisocyanate, a lysine-derived polyisocyanate with a vapor pressure substantially less than that of HDI.

Thus, the present invention relates to biocompatible and biodegradable polymers. Particularly, the invention relates to biocompatible and biodegradable polyurethane foams. In several embodiments, the present invention relates to injectable polyurethane foams, to methods and compositions for their preparation and to the use of such foams as scaffolds for bone tissue engineering.

In embodiments of the invention, porous scaffolds were synthesized by a one-shot foaming process, allowing for time to manipulate and inject the polymer, followed by rapid foaming and setting.

Synthetic biodegradable polymers are promising materials for bone tissue engineering. Many materials, including allografts, autografts, ceramics, polymers, and composites thereof are currently used as implants to repair damaged bone. Although autograft bone has the best capacity to stimulate healing of bone defects, explantation both introduces additional surgery pain and also risks donor-site morbidity. Synthetic polymers are advantageous because they can be designed with properties targeted for a given clinical application. Polymer scaffolds must support bone cell attachment, proliferation, and differentiation. Tuning the degradation rate with the rate of bone remodeling is an important consideration when selecting a synthetic polymer. Another important factor is the toxicity of the polymer and its degradation products. Furthermore, the polymer scaffold must be dimensionally and mechanically stable for a sufficient period of time to allow tissue in-growth and bone remodeling.

Two-component reactive liquid polyurethanes designed for tissue repair have been disclosed. For example, U.S. Pat. No. 6,306,177, the disclosure of which is incorporate herein by reference, discloses a method for repairing a tissue site comprising the steps of providing a curable polyurethane composition, mixing the parts of the composition, and curing the composition in the tissue site wherein the composition is sufficiently flowable to permit injection by minimally invasive techniques and exhibits a tensile strength between 6,000 and 10,000 psi when cured. However, because this injectable polyurethane is non-porous and hard, tissue ingrowth is likely to be limited.

U.S. Pat. No. 6,376,742, the disclosure of which is incorporated herein by reference, discloses a method for in vivo tissue engineering comprising the steps of combining a flowable polymerizable composition including a blowing agent and delivering the resultant composition to a wound site via a minimally invasive surgical technique. U.S. Pat. No. 6,376,742 also discloses methods to prepare microcellular polyurethane implants as well as implants seeded with cells.

Bennett et al. prepared porous polyurethane implants for bone tissue engineering from isocyanate-terminated prepolymers, water, and a tertiary amine catalyst (diethylethanolamine). See, for example, Bennett S, Connolly K, Lee D R, Jiang Y, Buck D, Hollinger J O, Gruskin E A. Initial biocompatibility studies of a novel degradable polymeric bone substitute that hardens in situ. Bone 1996; 19(1, Supplement):101S-107S; U.S. Pat. Nos. 5,578,662, 6,207,767 and 6,339,130, the disclosures of which are incorporated herein by reference. The prepolymers were synthesized from lysine methyl ester diisocyanate (LDI) and poly(dioxanone-co-glycolide) from a pentaerythritol initiator and then combined with either hydroxyapatite or tricalcium phosphate to form a putty. Water and a tertiary amine were added to the putty prior to implantation in rats. The putty did not elicit an adverse tissue response following implantation.

Zhang et al. prepared biodegradable polyurethane foams from LDI, glucose, and poly(ethylene glycol). Zhang J, Doll B, Beckman E, Hollinger J O. A biodegradable polyurethane-ascorbic acid scaffold for bone tissue engineering. J. Biomed. Mater. Res. 2003; 67A(2):389-400; Zhang J, Doll B, Beckman J, Hollinger J O. Three-dimensional biocompatible ascorbic acid-containing scaffold for bone tissue engineering. Tissue Engineering 2003; 9(6):1143-1157; Zhang J-Y, Beckman E J, Hu J, Yuang G-G, Agarwal S, Hollinger J O. Synthesis, biodegradability, and biocompatibility of lysine diisocyanate-glucose polymers. Tissue Engineering 2002; 8(5):771-785; and Zhang J-Y, Beckman E J, Piesco N J, Agarwal S. A new peptide-based urethane polymer: synthesis, biodegradation, and potential to support cell growth in vitro. Biomaterials 2000; 21:1247-1258., the disclosures of which are incorporated herein by reference. The foams were synthesized by reacting isocyanate-terminated prepolymers with water in the absence of catalysts. The polyurethane foams supported the attachment, proliferation, and differentiation of bone marrow stromal cells in vitro and were non-immunogenic in vivo. Bioactive foams were also prepared by adding ascorbic acid to the water prior to adding the prepolymer. As the polymer degraded, ascorbic acid was released to the matrix, resulting in enhanced expression of osteogenic markers such as alkaline phosphatase and Type I collagen.

Published PCT international patent application WO 2004/009227 A2, the disclosure of which is incorporated herein by reference, claims a star prepolymer composition suitable as an injectable biomaterial for tissue engineering. The prepolymer is the reaction product of a diisocyanate and a starter molecule having a molecular weight preferably less than 400 Da. Porous scaffolds were prepared by adding low levels (e.g., <0.5 parts per hundred parts polyol) of water.

Copending Published US Patent Application No. 2005/0013793 (U.S. patent application Ser. No. 10/759,904), the disclosure of which is incorporated herein by reference, discloses, inter alia, a biocompatible and biodegradable polyurethane composition including at least one biologically active component with an active hydrogen atom capable of reacting with isocyanates. As the polyurethane degrades in vivo, the bioactive component is released to the extracellular matrix where it is, for example, taken up by cells.

Published PCT international application WO 2006/055261, the disclosure of which is incorporated herein by reference, discloses a method of synthesizing of a biocompatible and biodegradable polyurethane foam includes the steps of: mixing at least one biocompatible polyol, water, at least one stabilizer, and at least one cell opener, to form a resin mix; contacting the resin mix with at least one polyisocyanate to form a reactive liquid mixture; and reacting the reactive liquid mixture form a polyurethane foam. The polyurethane foam is preferably biodegradable within a living organism to biocompatible degradation products. At least one biologically active molecule having at least one active hydrogen can be added to form the resin mix.

Also, WO 2009/026387 discloses additional aspects concerning the one-shot process, and is incorporated herein by reference.

While materials such as those described above are useful for bone tissue engineering, it is desirable to improve certain properties associated with injectable polyurethane scaffolds. Highly porous (e.g., >80% or even >85%), fast-rising (e.g., <30 minutes) conventional polyurethane foams have been manufactured commercially for years. For example, Ferrari and co-workers' in Ferrari R J, Sinner J W, Bill J C, Brucksch W F. Compounding polyurethanes: Humid aging can be controlled by choosing the right intermediate. Ind. Eng. Chem. 1958; 50(7):1041-1044, and U.S. Pat. No. 6,066,681, the disclosures of which is incorporated herein by reference, disclose methods for preparation of polyurethane foams from diisocyanates and polyester polyols. Catalysts, including organometallic compounds and tertiary amines, are added to balance the gelling (reaction of isocyanate with polyol) and blowing (reaction of isocyanate with water) reactions. Stabilizer, such as polyethersiloxanes and sulfated castor oil, are added to both emulsify the raw materials and stabilize the rising bubbles.

Although progress has been made in the development of biocompatible and biodegradable polymers, it remains desirable to develop biocompatible and biodegradable polymers, methods of synthesizing such polymers, implantable devices comprising such polymers and methods of using such polymers.

### SUMMARY OF THE INVENTION

In one aspect, the present invention provides a method of synthesizing of a biocompatible and biodegradable polyurethane foam including the steps of: mixing at least one biocompatible polyol, water, at least one stabilizer, and mineralized bone particles, to form a resin mix; contacting the resin mix with at least one polyisocyanate to form a reactive liquid mixture; and reacting the reactive liquid mixture form a polyurethane foam. In embodiments, of the present invention, the polyisocyanate is a tri-functional isocyanate.

The polyurethane foam is preferably biodegradable within a living organism to biocompatible degradation products. At least one biologically active molecule having at least one active hydrogen can be added to form the resin mix.

To promote transport of cells, fluids, and signaling molecules, the foams can have a porosity greater than 50 vol-%. The porosity ε, or void fraction, is calculated as shown in WO '261 and below.

In several embodiments, at least one catalyst is added to form the resin mix. Preferably, the catalyst is non-toxic (in a concentration that may remain in the polymer).

In several embodiments, the polyol is biocompatible and has a hydroxyl number in the range of approximately 50 to 1600. The polyol can, for example, be a biocompatible and polyether polyol or a biocompatible polyester polyol. In several embodiments, the polyol is a polyester polyol synthesized from at least one of ε-caprolactone, glycolide, or DL-lactide.

Water can, for example, be present in the resin mix in a concentration in a range of approximately 0.1 to 4 parts per hundred parts polyol.

The stabilizer is preferably nontoxic (in a concentration remaining in the polyurethane foam) and can include non-ionic surfactant or an anionic surfactant.

The polyisocyanate can, for example, be a biocompatible aliphatic polyisocyanate derived from a biocompatible polyamine compound (for example, amino acids). Examples of suitable aliphatic polyisocyanates include lysine methyl ester diisocyanate, lysine triisocyanate; 1,4-diisocyanatobutane, or hexamethylene diisocyanate. As stated above, embodiments of the present invention comprises tri-functional isocyanate.

The polyurethane foams of the present invention are preferably synthesized without aromatic isocyanate compounds. The method of the present invention can also include the step of placing the reactive liquid mixture in a mold in which the reactive liquid mixture is reacted to form the polyurethane foam.

The invention can, for example, provide dimensionally stable, high porosity, injectable, biocompatible, biodegradable and (optionally) biologically active polyurethane foams. The open-pore content can be sufficiently high to prevent shrinkage of the foam. The foams of the present invention can, for example, support the attachment and proliferation of cells in vitro and are designed to degrade to and release biocompatible components in vivo. In that regard, the present invention also provides scaffolds for cell proliferation/growth comprising a polyurethane polymer as set forth above and/or fabricated using a synthetic method as described above.

The polyurethane compositions of the present invention are useful for a variety of applications, including, but not limited to, injectable scaffolds for bone tissue engineering and drug and gene delivery. The compositions of the present invention can, for example, be applied to a surface of a bone, deposited in a cavity or hole formed in a bone, injected into a bone or positioned between two pieces of bone. The compositions can be injected through the skin of a patient to, for example, fill a void, cavity or hole in a bone using, for example, a syringe. Likewise, the compositions of the present invention can be molded into any number of forms outside of the body and placed into the body. For example, the compositions of the present invention can be formed into a plate, a screw, a prosthetic element, a molded implant etc.

The invention encompasses methods and compositions for preparing biocompatible and biodegradable polyurethane foams that are dimensionally stable.

The invention also encompasses methods of synthesizing of a biocompatible and biodegradable polyurethane foam comprising the steps of: mixing at least one biocompatible polyol, PEG, water, at least one stabilizer, and at least one pore opener, to form a resin mix; contacting the resin mix with at least one HDIt polyisocyanate to form a reactive liquid mixture; and reacting the reactive liquid mixture form a polyurethane foam. In this embodiment, the polyurethane foam being biodegradable within a living organism to biocompatible degradation products. In other aspects of this embodiment, at least one catalyst is added to form the resin mix.

The density of this embodiment may be at least 90 kg m−3. In other aspects, the density may be at least from about 75 to about 125 kg m−3.

Aspects of this embodiment may further comprise PEG. The PEG may be present in an amount of about 50% or less w/w. In other aspects, the PEG may be present in an amount of about 30% or less w/w. The PEG may have a MW of 600, for example. The PEG may be added in an amount up to about 60% polyol component.

In scaffolds of the present invention, the glass transition temperature may be in a range of about −50 to about 20. In other aspects of the present invention, the glass transition temperature is in a range of about −20 to about 10.

The porosity of the polyurethane scaffolds of the present invention may be, for example, greater than 70 (vol-%). In other aspects, the porosity may be from about 90 to about 95 (vol-%).

The pore size of scaffolds of the present invention may be, for example, about 100-1000 μm. In other aspects, the pore size may be about 200-500 μm.

The polyurethane scaffolds of the present invention may be comprised of at least one growth factor. Examples of the growth factors are PDGF, VEGF, and BMP-2.

The polyurethane scaffolds of the present invention may optionally further comprise a stabilizer, such as a stabilizer chosen from a polyethersiloxane, sulfonated caster oil, and sodium ricinoleicsulfonate.

The polyurethane scaffolds of the present invention may further comprise a biologically active agent. One example of a biologically active agent is demineralized bone particles. Other examples include agents chosen from enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, and prions.

In other aspects of the present invention, the PEG may be present in an amount of about 40 wt % or less. In others, the PEG is present in an amount of about 30 wt % or less.

The present invention, along with the attributes and attendant advantages thereof, will best be appreciated and understood in view of the following detailed description taken in conjunction with the accompanying drawings.

More specifically, one aspect of the present invention is a method of synthesizing of a biocompatible and biodegradable allograft bone/polyurethane composite foam comprising the steps of mixing at least one biocompatible polyol, water, at least one catalyst, at least one stabilizer, and mineralized bone powder to form a resin mix; contacting the resin mix with allograft bone particles to form a reactive paste; contacting the reactive paste with at least one isocyanate to form a reactive liquid mixture; and reacting the reactive liquid mixture form a composite allograft bone/polyurethane composite foam. The polyurethane foams of the present invention are biodegradable within a living organism to biocompatible degradation products. In certain embodiments of the present invention, the allograft bone particles may comprise mineralized bone particles. In others, the allograft bone component comprises demineralized bone matrix (DBM) in excess of 38 wt %.

In other embodiments, the resin mix comprises PGE.

In other embodiments, the catalyst is a tertiary amine catalyst.

In other embodiments, the stabilizer is turkey red oil.

In other embodiments, the isocyanate is a lysine-derived isocyanate. Also, the isocyanate may be an aliphatic isocyanate.

Other aspects of the present invention include biodegradable polyurethane scaffolds that comprise mineralized bone powder; at least one lysine-derived isocyanate; at least one polyol; wherein the porosity of said scaffold is from about 20 to about 70% and at least 50% of the pores are interconnected with another pore.

Other aspects of the present invention include a biodegradable polyurethane scaffold, comprising demineralized bone matrix in an amount greater than about 38.1 wt %; water in an about greater than about 1.6 pphp; at least one lysine-derived isocyanate; and at least one polyol. The porosity of these scaffolds may range from about 20 to about 70% and at least 50% of the pores are interconnected with another pore.

Yet another aspect of the present invention is a method of treating a bone injury site comprising the methods and scaffolds disclosed herein.

### DESCRIPTION OF THE INVENTION

An embodiment of the present invention is a biocompatible and biodegradable polymer-bone composition. Particularly, embodiments of the present invention include biocompatible and biodegradable polymeric foams. In several embodiments, these foams are injectable. Related embodiments of the present invention include methods and compositions for their preparation and the use of these foams as scaffolds for bone tissue engineering.

The polyurethane foam is preferably biodegradable within a living organism to biocompatible degradation products. At least one biologically active molecule having at least one active hydrogen can be added to form the resin mix.

An embodiment of the present invention is an injectable, biodegradable polyurethane (PUR) foam with mineralized bone particles to promote fracture healing and bone regeneration. The foams are made by reactive liquid molding of three components: (a) a polyisocyanate; (b) a resin comprised of a poly(ε-caprolactone-co-glycolide-co-lactide) polyol, water, an amine catalyst, and a stabilizer; and (c) an allograft bone component, which can be added to either the polyisocyanate or the resin. If the allograft bone is added to the resin, its contact with water should be minimized. The scaffolds provide structural support for healing bone, while the presence of the bone filler enhances the osteoconductive capacity of the foams. We have produced foams with as much as 40 wt-% demineralized bone matrix (DBM) and 75 wt-% mineralized bone particles (MBP).

Prior materials have been made that comprise lysine triisocyanate and demineralized bone matrix (DBM) at 18 and 38 wt %. DBM is made from allograft bone by completely demineralizing the material, leaving a sponge-like material. These materials were made with the calcium stearate pore opener in the composition, and the DBM was added to the resin with the water added. This composition is not desirable because, in part, it is difficult to consistently reproduce.

Embodiments of the present invention comprise polyisocyanate. Typically the polyisocyanate may be added to the resin, but the water is be added later or it will be absorbed by the bone, which is hygroscopic.

One embodiment of a reactive liquid molding process of the present invention for preparing the polyurethane foam is contacting an aliphatic polyisocyanate (or an isocyanate-terminated prepolymer) component (component 1) with a resin mix component (component 2) comprising at least one polyol, PEG, water, and optionally at least one cell opener. Preferably at least one catalyst is also present in the resin mix component. In several embodiments, one or more bioactive components are present in the resin mix component. The resin mix of component 2 is mixed with the polyisocyanate or multi-functional isocyanate compounds (that, compounds have a plurality of isocyanate function groups) of component 1 to form a reactive liquid composition. The reactive liquid composition can, for example, be cast into a mold either inside or outside the body where it cures to form a porous polyurethane. Thus, as used herein the term “mold” refers generally to any cavity or volume in which the reactive liquid composition is placed, whether that cavity or volume is formed manually or naturally outside of a body or within a body. See, for example, WO 2006/055,261.

In embodiments of the invention, the value of the index is in the range of approximately 80 to 140 and, more preferably, in the range of approximately 100 to 130.

In embodiments of the present invention, the hydroxyl number of the polyol/polyol blend is in the range of approximately 50 to 1600.

Polyester polyols are particularly suitable for use in the present invention because they hydrolyze in vivo to non-toxic, biocompatible degradation products. In several preferred embodiments of the present invention, the polyol is a polyester polyol or blend thereof having a hydroxyl number preferably in the range of approximately 80 to 420. Polyester polyols suitable for use in the present invention can, for example, be synthesized from at least one of the group of monomers including ε-caprolactone, glycolide, or DL-lactide.

Water reacts with polyisocyanate to form a disubstituted urea and carbon dioxide, which acts as a blowing agent. This reaction is referred to as the blowing reaction and results in a porous structure. The concentration of water in the resin mix affects the porosity and pore size distribution. To promote the presence of inter-connected pores, the concentration of water in the resin mix is preferably in the range of approximately 0.1 to 5 parts per hundred parts polyol (pphp) and, more preferably, in the range of approximately 0.5 to 3 pphp.

To form a dimensionally stable and highly porous foam, the rates of the gelling and blowing reactions are preferably balanced. This balance of rates can be accomplished through the use of catalysts, which can, for example, include an organometallic urethane catalyst, a tertiary amine urethane catalyst or a mixture thereof. In general, suitable catalysts for use in the present invention include compounds known in the art as effective urethane blowing and gelling catalysts, including, but not limited to, stannous octoate, organobismuth compounds (e.g., Coscat 83), triethylene diamine, bis(dimethylaminoethyl)ether, and dimethylethanolamine. Tertiary amine catalysts are preferred as a result of their generally lower toxicity relative to, for example, organometallic compounds. Triethylene diamine, which functions as both a blowing and gelling catalyst, is particularly preferred. Concentrations of catalyst blend in the resin mix are preferably in the range or approximately 0.1 to 5 pphp and, more preferably, in the range of approximately 0.5 to 5.0 pphp and, even more preferably, in the range of approximately 1 to 5 or in the range of approximately 1 to 4.

To promote transport of cells, fluids, and signaling molecules, the foams can have a porosity greater than 50 vol-%. The porosity c, or void fraction, is calculated as shown in WO '261.

In embodiments of the present invention, the polyisocyanate is a tri-functional isocyanate. Additionally, the polyisocyanate can, for example, be a biocompatible aliphatic polyisocyanate derived from a biocompatible polyamine compound (for example, amino acids). Examples of suitable aliphatic polyisocyanates include lysine methyl ester diisocyanate, lysine triisocyanate, 1,4-diisocyanatobutane, or hexamethylene diisocyanate. As stated above, embodiments of the present invention comprises tri-functional isocyanate.

As stated above, the resin comprises a poly(ε-caprolactone-co-glycolide-co-lactide) polyol, water, an amine catalyst, and a stabilizer.

In several embodiments, at least one catalyst is added to form the resin mix. Preferably, the catalyst is non-toxic (in a concentration that may remain in the polymer).

The catalyst can, for example, be present in the resin mix in a concentration in the range of approximately 0.5 to 5 parts per hundred parts polyol and, preferably in the range of approximately 1 to 5. The catalyst can, for example, be an organometallic compound or a tertiary amine compound. In several embodiments the catalyst includes stannous octoate, an organobismuth compound, triethylene diamine, bis(dimethylaminoethyl)ether, or dimethylethanolamine. An example of a preferred catalyst is triethylene diamine.

In several embodiments, the polyol is biocompatible and has a hydroxyl number in the range of approximately 50 to 1600. The polyol can, for example, be a biocompatible and polyether polyol or a biocompatible polyester polyol. In several embodiments, the polyol is a polyester polyol synthesized from at least one of ε-caprolactone, glycolide, or DL-lactide.

Water can, for example, be present in the resin mix in a concentration in a range of approximately 0.1 to 4 parts per hundred parts polyol.

The stabilizer is preferably nontoxic (in a concentration remaining in the polyurethane foam) and can include non-ionic surfactant or an anionic surfactant. The stabilizer can, for example, be a polyethersiloxane, a salt of a fatty sulfonic acid or a salt of a fatty acid, in the case that the stabilizer is a polyethersiloxane, the concentration of polyethersiloxane in the resin mix can, for example, be in the range of approximately 0.25 to 4 parts per hundred polyol. Polyethersiloxane stabilizer are preferably hydrolyzable. In the case that the stabilizer is a salt of a fatty sulfonic acid, the concentration of the salt of the fatty sulfonic acid in the resin mix is in the range of approximately 0.5 to 5 parts per hundred polyol. In the case that the stabilizer is a salt of a fatty acid, the concentration of the salt of the fatty acid in the resin mix is in the range of approximately 0.5 to 5 parts per hundred polyol. Examples of suitable stabilizers include a sulfated castor oil or sodium ricinoleicsulfonate.

Foam stabilizers can be added to the resin mix of the present invention to, for example, disperse the raw materials, stabilize the rising carbon dioxide bubbles, and/or control the pore size of the foam. Although there has been a great deal of study of foam stabilizers (sometimes referred to herein as simply “stabilizers”) the operation of stabilizers during foaming is not completely understood. Without limitation to any mechanism of operation, it is believed that stabilizers preserve the thermodynamically unstable state of a foam during the time of rising by surface forces until the foam is hardened. In that regard, foam stabilizers lower the surface tension of the mixture of raw materials and operate as emulsifiers for the system. Stabilizers, catalysts and other polyurethane reaction components are discussed, for example, in Oertel, Günter, ed., Polyurethane Handbook, Hanser Gardner Publications, Inc. Cincinnati, Ohio, 99-108 (1994). A specific effect of stabilizers is believed to be the formation of surfactant monolayers at the interface of higher viscosity of the bulk phase, thereby increasing the elasticity of the surface and stabilizing expanding foam bubbles.

Stabilizers suitable for use in the present invention include, but are not limited to, non-ionic surfactants (e.g., polyethersiloxanes) and anionic surfactants (e.g., sodium or ammonium salts of fatty sulfonic acids or fatty acids). Polyethersiloxanes, sulfated castor oil (Turkey red oil), and sodium ricinoleicsulfonate are examples of preferred stabilizers for use in the present invention. In the case of polyethersiloxane stabilizers, the concentrations of polyethersiloxane stabilizer in the resin mix is preferably in the range of approximately 0.25 to 4 pphp and, more preferably, in the range of approximately 0.5 to 3 pphp. Preferably, polyethersiloxane compounds for use in the present invention are hydrolyzable. In the case of stabilizers including salts of fatty sulfonic acid and/or salts of fatty acid, the concentration of salts of a fatty sulfonic acid and/or salts of a fatty acid in the resin mix is preferably in the range of approximately 0.5 to 5 pphp and, more preferably, in the range of approximately 1 to 3 pphp.

The index of the foam, as defined by:

INDEX=100×number of NCO equivalents/number of OH equivalents

and can be in the range of approximately 80 to 140.

The invention can, for example, provide dimensionally stable, high porosity, injectable, biocompatible, biodegradable, and biologically active bone/polyurethane composite foams. The open-pore content can be sufficiently high to prevent shrinkage of the foam. The foams of the present invention can, for example, support the attachment and proliferation of cells in vitro and are designed to degrade to and release biocompatible components in vivo. In that regard, the present invention also provides scaffolds for cell proliferation/growth comprising a polyurethane polymer as set forth above and/or fabricated using a synthetic method as described above.

Typically, the biodegradable compounds of the present invention degrade by hydrolysis. As used herein, the term “biocompatible” refers to compounds that do not produce a toxic, injurious, or immunological response to living tissue (or to compounds that produce only an insubstantial toxic, injurious, or immunological response). The term nontoxic as used herein generally refers to substances or concentrations of substances that do not cause, either acutely or chronically, substantial damage to living tissue, impairment of the central nervous system, severe illness, or death. Components can be incorporated in nontoxic concentrations innocuously and without harm. As used herein, the term “biodegradable” refers generally to the capability of being broken down in the normal functioning of living organisms/tissue (preferably, into innocuous, nontoxic or biocompatible products).

The polyurethane compositions of the present invention are useful for a variety of applications, including, but not limited to, injectable scaffolds for bone tissue engineering and drug and gene delivery. The compositions of the present invention can, for example, be applied to a surface of a hone, deposited in a cavity or hole formed in a bone, injected into a bone or positioned between two pieces of bone. The compositions can be injected through the skin of a patient to, for example, fill a void, cavity or hole in a bone using, for example, a syringe. Likewise, the compositions of the present invention can be molded into any number of forms outside of the body and placed into the body. For example, the compositions of the present invention can be formed into a plate, a screw, a prosthetic element, a molded implant etc.

The invention encompasses methods and compositions for preparing biocompatible and biodegradable polyurethane foams that are dimensionally stable.

Embodiments of the present invention include synthesizing foams with lysine diisocyanate (LDI) and DBM or MBP. The foam formulations can be adjusted to allow for variations in the desired foam properties: higher wt-% of incorporated bone particles, and delay of the foam gel time to provide more time for injection and manipulation. In vitro experiments have demonstrated that these scaffolds are not cytotoxic, and that they facilitate cell infiltration, proliferation, and differentiation. In vivo, implantation in a rat wound healing model have shown integration into the surrounding tissue, efficient wound healing, production of new collagen matrix, and biodegradation of the material, with minimal inflammatory response. In addition, implants in a rat tibial defect, both with and without MBP, exhibited cellular infiltration and mineralization.

The scaffolds of the present invention provide a significant improvement over current bone graft and cement treatments of large fractures. They are both biodegradable and resorbable, so it can minimize total surgery time and invasiveness for patients. A benefit of the reactive liquid molding synthesis of the scaffolds of the present invention is that it allows them to be injectable and therefore minimally invasive during implantation. In addition, embodiments of the present invention can expand to fill the contours of the fracture site, enhancing bone-scaffold contact and fixation. Lastly, embodiments of the present invention have achieved higher loading levels of bone particles into the scaffolds.

Biologically active agents can optionally be added to the resin mix. As used herein, the term “bioactive” refers generally to an agent, a molecule, or a compound that affects biological or chemical events in a host. Bioactive agents may be synthetic molecules, biomolecules, or multimolecular entities and include, but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, and therapeutics. Cells and non-cellular biological entities, such as viruses, virenos, virus vectors, and prions can also be bioactive agents. Biologically active agents with at least one active hydrogen are preferred. Examples of chemical moieties with an active hydrogen are amine and hydroxyl groups. The active hydrogen reacts with free isocyanate in the reactive liquid mixture to form a covalent bond (e.g., urethane or urea linkage) between the bioactive molecule and the polyurethane. As the polyurethane degrades, the bioactive molecules are released and are free to elicit or modulate biological activity. The incorporation of biologically active components into biocompatible and biodegradable polyurethanes is discussed in some detail in US Patent Application No. 2005/0013793 (U.S. patent application Ser. No. 10/759,904).

After mixing the polyisocyanate and the resin mix, the resulting reactive liquid mixture is, for example, cast into a cavity or mold where the polyisocyanate reacts with the components of the resin mix having an active hydrogen to form a polyurethane foam. The reactive liquid mixture can be cast into a mold ex vivo and then implanted or can be cast directly onto a surface or into a cavity, volume or mold (for example, a wound) in the body.

### EXAMPLES

The following is presented to show examples of the present invention, and it not to be construed as being limiting thereof.

In the below examples, Allograft bone/polymer porous scaffolds were prepared by first mixing the hardener component, comprising the polyester triol, water, catalyst, stabilizer, and pore opener. The appropriate amount of bone was subsequently added to the polyol component and mixed for about 30-60 s. The isocyanate was then added and mixed for another 30-60 s prior to casting in a mold.

### Example 1

### Exemplary Formulation Ranges of the Present Invention

Polyol: about 100.0 pphp. This may be a polyester triol with a molecular weight of 900 g/mol and a backbone that includes about 50-70% caprolactone, about 20-40% glycolide, and about 5-20% DL-lactide. Other backbone compositions and molecular weights are possible. MW range: 300-3000, preferred 450-1800, particularly preferred 450-1200 g/mol.
Water: The desired water content depends on which filler is used and whether a pore opener (e.g., calcium stearate) is used. For MBP, calcium stearate is not required and the water content ranges from about 0-5 pphp, preferably 1-3 pphp, more preferably 1.5-2.5 pphp. For DBM calcium stearate is required, and the water content ranges from about 0-12 pphp, preferably 1.6-10 pphp, more preferably 2.5-10 pphp.
Tegoamin33: The desired Tegoamin33 catalyst ranges from about 1.5-6 pphp, preferably 2.5-5 pphp, more preferably 2.5-4.5 pphp.
Turkey Red Oil: about 1.2-1.8 pphp
Filler: Either mineralized bone particles (MBP) or demineralized bone particles (DBM) can be used. For MBP, the range is 25-75 wt %, preferably 40-60 wt %. For DBM the content is >38%, preferably 38.1-45%. By increasing the water >2.5 pphp, preferably >5 pphp, it is possible to prepare DBM porous scaffolds with >38 wt % DBM. Isocyanate: Lysine Diisocyanate (LDI)

### Example 2

### Sample Formulations of MBP Foams of the Present Invention

| Component | 1 | 2 | 3 | 4 | 5 | 6 |
|---|---|---|---|---|---|---|
| T6C3G1L900 | 100 | 100 | 100 | 100 | 100 | 100 |
| (polyester triol with | | | | | | |
| 60% caprolactone, | | | | | | |
| 30% glycolide, and | | | | | | |
| 10 lactide; 900 g/mol | | | | | | |
| mol wt.) | | | | | | |
| Water | 1.5 | 2.5 | 2.5 | 2.5 | 2.5 | 1.5 |
| TEGOAMIN 33 | 3.0 | 3.0 | 3.0 | 3.0 | 3.0 | 4.5 |
| Turkey Red Oil | 1.5 | 1.5 | 1.5 | 1.5 | 1.5 | 1.5 |
| MBP | 252 | 274 | 339 | 425 | 546 | 80 |
| (wt % MBP) | 60 | 60 | 65 | 70 | 75 | 26.4 |
| LDI | 221.8 | 320.1 | 320.1 | 184.9 | 184.9 | 192.7 |

In the above table, 1-6 are Example numbers, and Units are pphp (parts per hundred parts polyol).

### Example 3

### LDI Scaffolds Incorporating 40 wt % Demineralized Bone Matrix (DBM)

40 wt % DBM

| Component | Pphp |
|---|---|
| T6C3G1L900 | 100 |
| Water | 10 |
| TEGOAMIN 33 | 3.0 |
| Turkey Red Oil | 1.5 |
| Calcium stearate | 4.0 |
| DBM | 200 |
| (wt % DBM) | 40 |
| LDI | 186.7 |

### Example 4

A MBP/polyurethane composite foam was synthesized from Desmodur DN3300A (a trimer based on hexamethylene diisocyanate (HDI), supplied by Bayer MaterialScience, LLC, Pittsburgh, Pa.) and a polyester triol with a molecular weight of 900 g/mol and backbone 60% caprolactone, 30% lactide, and 10% glycolide. The composites were implanted into plug defects in the tibiae of athymic rats. Animals were sacrificed at 3 weeks the tibiae were extracted and embedded in polymethyl methacrylate for ground section histology. 50-μm sections were cut and stained with toluidene blue to visualize new bone formation. The figures show the histology at magnifications of 1×, 1.25×, 2.5×, and 5×. Bone marrow cells infiltrating the implant appear brown, unresorbed allograft (MBP) stains blue, and new bone formation appears dark blue to black in these sections. Unresorbed MBP can be distinguished from new bone formation based on its sharp edges and corners, compared to the more rounded edges and corners associated with new bone formation.

The invention thus being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification, including the Example, be considered as exemplary only, and not intended to limit the scope and spirit of the invention.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references, specifically including those listed below, are incorporated herein by reference.

Throughout this application, various publications are referenced. All such references, specifically including those listed below, are incorporated herein by reference.

### REFERENCES

J. S. Temenoff and A. G. Mikos. Injectable biodegradable materials for orthopedic tissue engineering. Biomaterials 21: 2405-2412 (2000).
P. Gunatillake, R. Mayadunne, R. Adhikari, and M. R. El-Gewely. Recent developments in biodegradable synthetic polymers, Biotechnology Annual Review, Vol. Volume 12, Elsevier, 2006, pp. 301-347.
S. L. Bourke and J. Kohn. Polymers derived from the amino acid -tyrosine: polycarbonates, polyarylates and copolymers with poly(ethylene glycol). Advanced Drug Delivery Reviews 55: 447-466 (2003).
S. I. Ertel and J. Kohn. Evaluation of a series of tyrosine-derived polycarbonates as degradable biomaterials. Journal of Biomedical Materials Research 28: 919-930 (1994).
C. Yu and J. Kohn. Tyrosine-PEG-derived poly(ether carbonate)s as new biomaterials: Part I: synthesis and evaluation. Biomaterials 20: 253-264 (1999).
A. D. Augst, H.-J. Kong, and D. J. Mooney. Alginate Hydrogels as Biomaterials. Macromolecular Bioscience 6: 623-633 (2006).
R. R. Chen and D. J. Mooney. Polymeric growth factor delivery strategies for tissue engineering. Pharmaceutical Research 20: 1103-1112 (2003).
N. Kipshidze, P. Chawla, and M. H. Keelan. Fibrin Meshwork as a Carrier for Delivery of Angiogenic Growth Factors in Patients With Ischemic Limb. Mayo Clinic Proceedings 74: 847-848 (1999).
V. Labhasetwar, J. Bonadio, S. Goldstein, W. Chen, and R. J. Levy. A DNA controlled-release coating for gene transfer: Transfection in skeletal and cardiac muscle. Journal of Pharmaceutical Sciences 87: 1347-1350 (1998).
K. Mizuno, K. Yamamura, K. Yano, T. Osada, S. Saeki, N. Takimoto, T. Sakurai, and Y. Nimura. Effect of chitosan film containing basic fibroblast growth factor on wound healing in genetically diabetic mice. Journal of Biomedical Materials Research Part A 64A: 177-181 (2003).
C. A. Simmons, E. Alsberg, S. Hsiong, W. J. Kim, and D. J. Mooney. Dual growth factor delivery and controlled scaffold degradation enhance in vivo bone formation by transplanted bone marrow stromal cells. Bone 35: 562-569 (2004).
S. A. Zawko, Q. Truong, and C. E. Schmidt. Drug-binding hydrogels of hyaluronic acid functionalized with β-cyclodextrin. Journal of Biomedical Materials Research Part A 9999: NA (2008).

M. D. Timmer, C. G. Ambrose, and A. G. Mikos. Evaluation of thermal- and photo-crosslinked biodegradable poly(propylene fumarate)-based networks. Journal of Biomedical Materials Research Part A 66A: 811-818 (2003).
C. W. Kim, R. Talac, L. Lu, M. J. Moore, B. L. Currier, and M. J. Yaszemski. Characterization of porous injectable poly-(propylene fumarate)-based bone graft substitute. Journal of Biomedical Materials Research Part A 9999: NA (2007).
J. P. Fisher, J. W. M. Vehof, D. Dean, J. P. van der Waerden, T. A. Holland, A. G.

Mikos, and J. A. Jansen. Soft and hard tissue response to photocrosslinked poly(propylene fumarate) scaffolds in a rabbit model. Journal of Biomedical Materials Research 59: 547-556 (2002).
S. J. Peter, L. Lu, D. J. Kim, and A. G. Mikos. Marrow stromal osteoblast function on a poly(propylene fumarate)/[beta]-tricalcium phosphate biodegradable orthopaedic composite. Biomaterials 21: 1207-1213 (2000).
S. J. Peter, S. T. Miller, G. Zhu, A. W. Yasko, and A. G. Mikos. In vivo degradation of a poly(propylene fumarate)/β-tricalcium phosphate injectable composite scaffold. Journal of Biomedical Materials Research 41: 1-7 (1998).
M. J. Yaszemski, R. G. Payne, W. C. Hayes, R. S. Langer, T. B. Aufdemorte, and A. G. Mikos. The Ingrowth of New Bone Tissue and Initial Mechanical Properties of a Degrading Polymeric Composite Scaffold. Tissue Engineering 1: 41-52 (1995).
D. H. R. Kempen, L. Lu, C. Kim, X. Zhu, W. J. A. Dhert, B. L. Curreir, and M. J.

Yaszemski. Controlled drug release from a novel injectable biodegradable microsphere/scaffold composite based on poly(propylene fumarate). Journal of Biomedical Materials Research, Part A 77A: 103-111 (2006).
I. C. Bonzani, R. Adhikari, S. Houshyar, R. Mayadunne, P. Gunatillake, and M. M. Stevens. Synthesis of two-component injectable polyurethanes for bone tissue engineering. Biomaterials 28: 423-433 (2007).
K. Gorna and S. Gogolewski. Preparation, degradation, and calcification of biodegradable polyurethane foams for bone graft substitutes. Journal of Biomedical Materials Research, Part A 67A: 813-827 (2003).
S. Guelcher, A. Srinivasan, A. Hafeman, K. Gallagher, J. Doctor, S. Khetan, S. McBride, and J. Hollinger. Synthesis, in vitro degradation, and mechanical properties of two-component poly(ester urethane)urea scaffolds: Effects of water and polyol composition. Tissue Engineering 13: 2321-2333 (2007).
J.-Y. Zhang, E. J. Beckman, J. Hu, G.-g. Yang, S. Agarwal, and J. O. Hollinger. Synthesis, biodegradability, and biocompatibility of lysine diisocyanate-glucose polymers. Tissue Engineering 8: 771-785 (2002).
R. Adhikari and P. A. Gunatillake. Biodegradable polyurethane/urea compositions, 2004.
M. Kitai, H. Ryuutou, T. Yahata, Y. Hara, H. Iwane, and Y. Soejima. Aliphatic triisocyanate compound, process for producing the same, and polyurethane resin made from the compound. In U. PTO (ed), Vol. 20020123644 A1 (U. PTO, ed), 2002.
S. A. Guelcher, V. Patel, K. M. Gallagher, S. Connolly, J. E. Didier, J. S. Doctor, and J. O. Hollinger. Synthesis and in vitro biocompatibility of injectable polyurethane foam scaffolds. Tissue Engineering 12: 1247-1259 (2006).
A. S. Sawhney and J. A. Hubbell. Rapidly degraded terpolymers of D,L-lactide, glycolide, and ε-caprolactone with increased hydrophilicity by coploymerization with polyethers. Journal of Biomedical Materials Research 24: 1397-1411 (1990).
ASTM-International. D3574-05. Standard test methods for flexible cellular materials—slab, bonded, and molded urethane foams, Vol. 08.02, 2007, pp. 360-368.
G. Evans, B. Atkinson, and G. Jameson. The Jameson Cell. In K. Matis (ed), Flotation Science and Engineering (K. Matis, ed), Marcel Dekker, New York, 1995, pp. 353.
ASTM-International. D695-02a. Standard Test Method for Compressive Properties of Rigid Plastics, Vol. 14.02, 2007.
G. Oertel. Polyurethane Handbook, Hanser Gardner Publications, Berlin, 1994.
R. Eriksson, T. Albrektsson, and B. Magnusson. Assessment of bone viability after heat trauma. A histological, histochemical and vital microscopic study in the rabbit. Scandinavian Journal of Plastic and Reconstructive Surgery 18: 261-268 (1984).
P. J. Meyer, E. Lautenschlager, and B. Moore. On the setting properties of acrylic bone cement. Journal of Bone & Joint Surgery. American volume 55: 149-156 (1973).
J. E. Mark, E. Erman, and F. R. Eirich. Science and Technology of Rubber, Academic Press, Inc., San Diego, Calif. (1994).
O. Kramer, S. Hvidt, and J. D. Ferry. Dynamic Mechanical Properties. In J. E. Mark, E. Erman, and F. R. Eirich (eds), Science and Technology of Rubber (J. E. Mark, E. Erman, and F. R. Eirich, eds), Academic Press, Inc., San Diego, Calif., 1994.
S. Gogolewski, K. Gorna, and A. S. Turner. Regeneration of bicortical defects in the iliac crest of estrogen-deficient sheep, using new biodegradable polyurethane bone graft substitutes. Journal of Biomedical Materials Research, Part A 77A: 802-810 (2006).
J. Guan, K. L. Fujimoto, M. S. Sacks, and W. R. Wagner. Preparation and characterization of highly porous, biodegradable polyurethane scaffolds for soft tissue applications. Biomaterials 26: 3961-3971 (2005).
J.-Y. Zhang, E. J. Beckman, N. P. Piesco, and S. Agarwal. A new peptide-based urethane polymer: synthesis, biodegradation, and potential to support cell growth in vitro. Biomaterials 21: 1247-0.1258 (2000).
J. H. de Groot, R. de Vrijer, B. S. Wildeboer, C. S. Spaans, and A. J. Pennings. New biomedical polyurethane ureas with high tear strengths. Polymer Bulletin 38: 211-218 (1997).
G. A. Skarja and K. A. Woodhouse. Structure-property relationships of degradable polyurethane elastomers containing an amino acid-based chain extender. Journal of Applied Polymer Science 75: 1522-1534 (2000).
N. Tsutsumi, S. Yoshizaki, W. Sakai, and T. Kiyotsukuri. Nonlinear optical polymers. 1. Novel network polyurethane with azobenzene dye in the main frame. Macromolecules 28: 6437-6442 (1995).
S. Gogolewski and K. Gorna. Biodegradable polyurethane cancellous bone graft substitutes in the treatment of iliac crest defects. Journal of Biomedical Materials Research Part A 80A: 94-101 (2007).
K. Gorna and S. Gogolewski. Molecular stability, mechanical properties, surface characteristics and sterility of biodegradable polyurethanes treated with low-temperature plasma. Polymer Degradation and Stability 79: 475-485 (2003).
K. Gorna, S. Polowinski, and S. Gogolewski. Synthesis and characterization of biodegradable poly(ε-caprolactone urethane)s. I. Effect of the polyol molecular weight, catalyst, and chain extender on the molecular and physical characteristics. Journal of Polymer Science, Part A: Polymer Chemistry 40: 156-170 (2002).
J. Guan, M. S. Sacks, E. J. Beckman, and W. R. Wagner. Biodegradable poly(ether ester urethane)urea elastomers based on poly(ether ester) triblock copolymers and putrescine: synthesis, characterization and cytocompatibility. Biomaterials 25: 85-96 (2004).
P. Bruin, G. J. Veenstra, A. J. Nijenhuis, and A. J. Pennings. Design and synthesis of biodegradable poly(ester-urethane) elastomer networks composed of non-toxic building blocks. Die Makromolekulare Chemie, Rapid Communications 9: 589-594 (1988).
S. L. Elliott, J. D. Fromstein, J. P. Santerre, and K. A. Woodhouse. Identification of biodegradation products formed by L-phenylalanine based segmented polyurethaneureas. Journal of Biomaterials Science Polymer Edition 13: 691-711 (2002).
L. Lu, S. J. Peter, M. D. Lyman, H.-L. Lai, S. M. Leite, J. A. Tamada, S. Uyama, J. P. Vacanti, R. Langer, and A. G. Mikos. In vitro and in vivo degradation of porous poly(D-lactic-co-glycolic acid) foams. Biomaterials 21: 1837-1845 (2000).
J. Guan, J. J. Stankus, and W. R. Wagner. Biodegradable elastomeric scaffolds with basic fibroblast growth factor release. Journal of Controlled Release 120: 70-78 (2007).
J.-Y. Zhang, B. A. Doll, E. J. Beckman, and J. O. Hollinger. A biodegradable polyurethane-ascorbic acid scaffold for bone tissue engineering. Journal of Biomedical Materials Research, Part A 67: 389-400 (2003).

We claim:

1. A method of synthesizing of a biocompatible and biodegradable allograft bone/polyurethane composite foam comprising the steps of:
mixing at least one biocompatible polyol, water, at least one catalyst, at least one stabilizer, and mineralized bone powder to form a resin mix;
contacting the resin mix with allograft bone particles to form a reactive paste;
contacting the reactive paste with at least one isocyanate to form a reactive liquid mixture; and
reacting the reactive liquid mixture form a composite allograft bone/polyurethane composite foam;
the polyurethane foam being biodegradable within a living organism to biocompatible degradation products.

2. The method of claim 1, wherein the resin mix further comprises PEG.

3. The method of claim 1, wherein the catalyst is a tertiary amine catalyst.

4. The method of claim 1, where in the stabilizer is turkey red oil.

5. The method of claim 1, wherein the isocyanate is a lysine-derived isocyanate.

6. The method of claim 1, wherein the isocyanate is an aliphatic isocyanate.

7. The method of claim 1, wherein the allograft bone component comprises mineralized bone particles (MBP).

8. The method of claim 1, wherein the allograft bone component comprises demineralized bone matrix (DBM) in excess of 38 wt %.

9. The method of claim 1, wherein the polyol is a polyester triol.

10. A biodegradable polyurethane scaffold, comprising
mineralized bone powder;
at least one lysine-derived isocyanate;
at least one polyol; wherein
the porosity of said scaffold is from about 20 to about 70% and at least 50% of the pores are interconnected with another pore.

11. The polyurethane scaffold of claim 10, wherein the density is at least about 20%.

12. The polyurethane scaffold of claim 10, wherein the density is from about 20 to about 70%.

13. The polyurethane scaffold of claim 9, further comprising PEG.

14. The polyurethane scaffold of claim 12, wherein the PEG is present in an amount of about 50% or less w/w.

15. The polyurethane scaffold of claim 13, wherein the PEG is present in an amount of about 30% or less w/w.

16. The polyurethane scaffold of claim 15, wherein the polyester triol has a backbone that comprises caprolactone, glycolide, and DL-lactide.

17. The polyurethane scaffold of claim 10, wherein the lysine-derived isocyanate is lysine diisocyanate.

18. The polyurethane scaffold of claim 10, wherein the lysine-derived isocyanate is an aliphatic polyisocyanate.

19. The polyurethane scaffold of claim 10, wherein the pore size is about 100-1000 μm.

20. The polyurethane scaffold of claim 10, wherein the pore size is about 200-500 μm.

21. The polyurethane scaffold of claim 9, further comprising a stabilizer chosen from a polyethersiloxane, sulfonated caster oil, turkey red oil, and sodium ricinoleicsulfonate.

22. The polyurethane scaffold of claim 9, wherein the polyol is a polyester triol present in an amount of from about 10 to about 70 wt %.

23. The polyurethane scaffold of claim 9, wherein the polyol is a polyester triol present in an amount of from about 20 to about 60 wt %.

24. A biodegradable polyurethane scaffold, comprising
demineralized bone matrix in an amount greater than about 38.1 wt %;
water in an about greater than about 1.6 pphp;
at least one lysine-derived isocyanate;
at least one polyol; wherein
the porosity of said scaffold is from about 20 to about 70% and at least 50% of the pores are interconnected with another pore.

25. A method of treating a bone injury site, comprising:
preparing a biodegradable polyurethane foam by mixing at least one biocompatible polyol, water, at least one stabilizer, and mineralized bone powder to form a resin mix, contacting the resin mix with at least one isocyanate to form a reactive liquid mixture, and reacting the reactive liquid mixture form a polyurethane foam that is biodegradable within a living organism; and
contacting the biodegradable polyurethane foam with the injury site.
